(19) United States
(12) Patent Application Publication
(10) Pub. No.: US 2006/0106350 A1
(43) Pub. Date: May 18, 2006

(54) Apparatus and methods for treating undesired veins

(76) Inventor: Gregory A. Spitz, Aurora, IL (US)

Correspondence Address: CARDINAL LAW GROUP, Suite 2000, 1603 Orrington Avenue, Evanston, IL, 60201, US

(21) Appl. No.: 11/087,356

(22) Filed: Mar. 23, 2005

Related U.S. Application Data
(60) Provisional application No. 60/609,531, filed on Sep. 13, 2004.

(51) Int. Cl. A61M 31/00

(52) U.S. Cl. 604/258; 604/507

(57) ABSTRACT

A method of treating undesired veins includes inserting a needle into a segment of an undesired vein, injecting a sclerosing agent through the needle into the undesired vein to initiate occlusion of the vein, and removing the needle from the undesired vein. The method further includes inserting a needle of a fluid delivery system into the surrounding tissue of the undesired vein, and delivering a tumescent solution through the needle of the fluid delivery system into the surrounding tissue of the vein to increase compression to the vein. A surgical apparatus for treating undesired veins includes a fluid source to store the fluid and a fluid transmitting device. A fluid delivery tube having first and second lumens is coupled to the fluid transmitting device. A first needle is in fluid communication with the first lumen and a second needle is in fluid communication with the second lumen.

### RELATED APPLICATIONS

This application claims priority to U.S. Provisional Application No. 60/609,531, filed Sep. 13, 2004, titled “Apparatus and Methods for Treating Undesired Veins”, by Gregory Spitz, the entirety of which is incorporated herein by reference.

### FIELD OF THE INVENTION

The present invention relates generally to surgical apparatus and procedures. More particularly, it relates to methods and apparatus for treatment of veins, such as, varicose and spider veins, in a venous system of a patient.

### BACKGROUND

Spider and varicose veins are common conditions that occur in many humans and are typically found in the lower limbs of the human body. Spider veins generally consist of small, thin, dark-colored veins that lie close to the surface of the skin. They often have a web or sunburst pattern, but may also appear as random line segments. Varicose veins are larger veins in comparison to spider veins. Varicose veins usually have a blue or purple color and may protrude above the surface of the skin. These veins have usually lost their ability to carry blood back to the heart and blood often accumulates in these veins.

A number of factors can contribute to the development of varicose and spider veins, including heredity, obesity, posture, hormonal shifts, excessive heat, and standing or sitting for a long periods of time. Spider and varicose veins may cause patients to experience various symptoms, such as aching, burning, swelling, cramping, and itching, while more serious complications of varicose veins can include thrombophlebitis, dermatitis, hemorrhage and ulcers. If certain varicose veins are not treated, blood clots may form in the vein, and phlebitis or inflammation of the inside lining of the vein may occur. Even absent such symptoms, many patients seek medical treatment of varicose and spider veins for cosmetic reasons.

Various approaches have been developed to treat spider and varicose veins. These treatments include vein removal for severe cases and sclerotherapy for smaller varicose and spider veins lying close to the surface of the skin. In one vein removal technique, called stab avulsion phelbectomy, the entire vein or a portion of the vein is removed. In order to remove the vein, the surgeon typically makes one or more incisions in the skin of a patient. A hook is inserted into the incisions to grip or hook the vein to be removed. When the vein is grabbed, the vein is pulled though the surgical incision and severed. However, this procedure usually requires two surgeons to perform the procedure and takes about 2-3 hours to complete. In addition, this procedure usually requires multiple incisions in the patient in order to hook the affected vein. Furthermore, it is often difficult to completely remove the entire affected vein using this procedure.

Varicose veins can also be removed by a procedure commonly referred to as “stripping.” This procedure involves tearing out the vein axis using a wire. To remove a vein using a stripping procedure, incisions are made through the skin of the patient to provide access to the vein. The ends of the vein are then separated from other veins. A vein stripper, such as a wire, is then inserted into the lumen of the vein. The wire is then advanced through the vein and tied to the distal end of the vein. The wire is then pulled or retracted through the incision to extract the vein from the surrounding tissue. After the vein is completely removed from the leg, the incisions are closed.

However, this operation is typical performed under an anesthetic which can be general, loco-regional, peridural, or under neuroleptanalgesia. This procedure may also damage the collateral branches of the vein which may consequently bleed, giving rise to hematomas. This operation can also be painful and uncomfortable for patients in the hours and days following surgery. There may also be other complications with this technique, such as, blood loss, pain, infection, hematoma, nerve injury, and swelling.

In less complicated cases, elevation of the legs and use of support hosiery may be sufficient therapy to stop or slow the progression of the varicose veins. Alternatively, a technique called “sclerotherapy” may be used to treat varicose and spider veins. In this procedure, the affected veins are injected with a sclerosing solution. The sclerosing solution causes inflammation and subsequent sclerosis of the vein. The sclerosis results in localized scarring or closure of the veins, which forces re-routing of the blood away from the affected veins.

When treating veins with a sclerosing solution, the sclerosing solution may not stay in the desired portion of the vein being treated and may leak outside of the vein causing skin ulcerations. Hyper-pigmentation may also occur as a result of the leakage of a blood component, hemosiderin pigment, from the treated vein. The sclerosing solution can also cause inflammation in the region where the solution was injected. In order to limit postoperative inflammation, the medical practitioner usually applies compression to the treated area immediately after sclerosing solution has been injected. The patient is also usually required to wear support hosiery for 48 consecutive hours (night and day) after treatment and, during the waking hours for seven more days.

### SUMMARY OF THE INVENTION

In view of the above, the present invention provides methods and apparatus for treating undesired veins, such as, varicose and spider veins, in a venous system of a patient. The methods and apparatus provide improved techniques for treating spider and varicose veins using sclerotherapy techniques. The methods and apparatus introduce saline or tumescent solution into the tissue where the vein has been injected with a sclerosing solution. The introduction of the tumescent solution into the tissue causes the surrounding tissue to become swollen and firm, thereby compressing the vein and maintaining the sclerosing solution in the portion of the vein in which it was injected. As a result, the effect of the sclerosing solution is increased. The treated veins become less noticeable in a shorter period of time, and the patient has a faster recovery time with improved results. The tumescent solution also reduces hyper-pigmentation and inflammation of the tissue surrounding the vein and decreases any itching and pain to the patient. This method further reduces the amount of time that patient has to wear support hosiery and decreases the number of treatment sessions for the patient, thereby reducing the cost to the patient. The present invention also allows the medical practitioner to efficiently deliver the tumescent solution around the vein during the sclerotherapy procedure.

One method of treating undesired veins in accordance with the present invention includes the steps of inserting a needle of a syringe into a segment of an undesired vein, injecting a sclerosing agent through the needle into the segment of the undesired vein to initiate occlusion of the vein, and removing the needle of the syringe from the segment of the undesired vein. The method further includes the steps of inserting a needle of a fluid delivery system into the surrounding tissue of the undesired vein, and delivering a tumescent solution through the needle of the fluid delivery system into the surrounding tissue of the vein to increase compression to the undesired vein.

One surgical apparatus for treating veins in accordance with the present invention includes a fluid delivery system to deliver fluid into the tissue of a patient. The fluid delivery system includes a fluid source to store the fluid and a fluid transmitting device in fluid communication with the fluid source. A fluid delivery tube having first and second lumens is coupled to the fluid transmitting device. A first needle is in fluid communication with the first lumen and a second needle is in fluid communication with the second lumen. The fluid delivery system allows a medical practitioner to inject a tumescent solution into the tissue where the vein has been injected with a sclerosing agent.

The invention, together with further attendant advantages, will best be understood by reference to the following detailed description of the presently preferred embodiments of the invention, taken in conjunction with the accompanying drawings. The drawings have not been drawn to scale. It is to be understood that both the foregoing general description and the following detailed description are exemplary and explanatory and are intended to provide further explanation of the invention as claimed.

### BRIEF DESCRIPTION OF THE DRAWINGS

FIG. 1 is a diagrammatical view of a portion of an undesired vein in the legs of a patient being injected with a sclerosing agent;

FIG. 2 is a diagrammatical view of a fluid being introduced by a fluid delivery system into the surround tissue around of the undesired vein of FIG. 1; and

FIG. 3 is a perspective view of a fluid delivery system to inject fluid into the surrounding tissue of a vein in a venous system of a patient.

### DETAILED DESCRIPTION OF THE PRESENTLY PREFERRED EMBODIMENTS

Before explaining the preferred embodiment in detail, it should be noted that the invention is not limited in its application or use to the details of construction and arrangement of parts illustrated in the accompanying drawings and description, because the illustrative embodiment of the invention may be implemented or incorporated in other embodiments, variations and modifications, and may be practiced or carried out in various ways. Furthermore, unless otherwise indicated, the terms and expressions employed herein have been chosen for the purpose of describing the preferred embodiment of the present invention for the convenience of the reader and are not for the purpose of limitation.

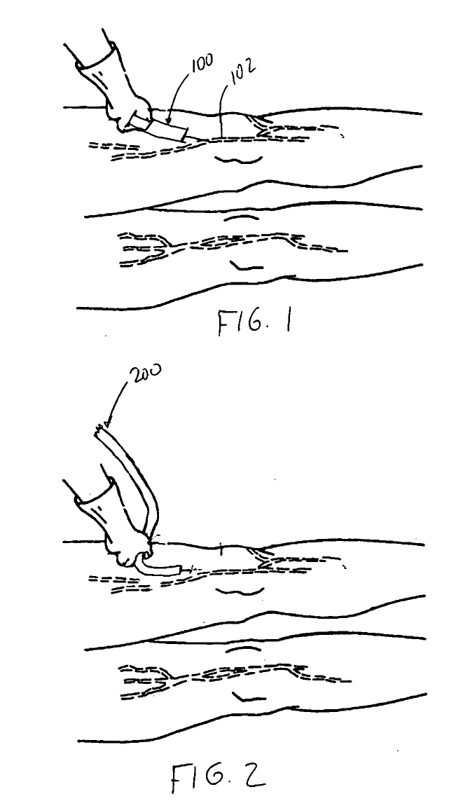

Referring now to the drawings in detail, and particularly to FIGS. 1-2, a surgical procedure for treating undesired veins, such as varicose and spider veins, of a patient will now be described. Although the procedure will be described in reference to a vein in a patient's leg, it will be recognized that the following procedure can be used to treat any suitable vein in a patient.

Prior to the treatment of the patient, a sclerosing solution is prepared by a medical practitioner for delivery into the undesired veins of the patient. The fluid can comprise, for example, hypertonic saline, sodium tetradecyl sulfate (“Sotradecol”), or aethoxyskerol (“Polidocanol”), but can be any suitable therapeutic substance or sclerosing solution. After the sclerosing solution has been prepared, a number of syringes are filled with the sclerosing solution. During a typical treatment session, 30-40 syringes are used to inject the sclerosing solution into the veins of the patient. Depending upon the concentration or strength of the sclerosing solution, more or less syringes may be used. The syringes preferably have fine needles to inject the sclerosing solution into the veins, thereby reducing any pain caused to the patients as a result of the needle being inserted through the patient's skin.

In addition to preparing of the sclerosing solution, a saline or tumescent solution is also prepared for delivery into the surrounding tissue of the vein where the sclerosing solution has been injected. The tumescent solution can comprise, for example, 50 cc of 1% Lidocaine, 2 cc of 1:1000 epinephrine (adrenaline), and 1 liter of saline, but can be any suitable solution. As further described below, a fluid delivery system is used to introduce the tumescent solution into the patient.

Referring now to FIG. 1, a partial perspective view of a portion of the leg showing the skin layer and segments of undesired vein are illustrated. In this procedure, the patient is positioned to expose the veins to be treated. If desired, the area in close proximity of the undesired vein can be anesthetized.

Once the medical practitioner locates and identifies the vein to be treated, the medical practitioner inserts the needle of one of the syringes 100 through the skin or tissue of the patient and into the portion of the vein 102. If the medical practitioner observes little or no change in appearance in the vein, then the needle may not be properly located in the vein. The needle is then withdrawn and inserted at another location in the vein before the sclerosing solution is injected.

Once the needle is properly inserted into the vein, the medical practitioner presses the plunger of the syringe 100 to inject the sclerosing solution through the needle directly into the vein 102. After the medical practitioner injects the sclerosing solution into the vein of the patient, the medical practitioner removes the needle from the patient. This procedure is repeated until substantially all the desired varicose and spider veins have been treated. Approximately one injection of the solution is usually administered for every inch of the affected veins, and multiple injections may be administered during a treatment session. Once the sclerosing agent is inside the vein, the sclerosing agent begins to irritate the internal lining of the vein, causing inflammation and, eventually, blood clotting and scarring that permanently blocks the vein.

After the sclerosing solution has been injected into the desired segments of the veins, the medical practitioner inserts a needle of the fluid delivery system 200, as shown in FIG. 2 and further described below, through skin or tissue of the patient and into the surrounding tissue of the vein where the sclerosing agent has been injected. Once the needle is inserted, the medical practitioner activates the surgical delivery system 200 to introduce the tumescent solution into the surround tissue. The tumescent solution is introduced around the outside wall of the vein and under the skin of the patient. The medical practitioner continues to delivery the tumescent solution until the skin becomes swollen and firm, thereby compressing the vein to increase occlusion and causing blood to be rerouted away from the treated vein.

The tumescent solution also provides anesthetic benefits to reduce the pain to the patient. As will be further described below, the medical practitioner may use a small needle to begin to introduce the tumescent solution into the surrounding tissue of the treated vein. After the tumescent solution causes the area to become locally anesthetized, the medical practitioner can use a larger needle to introduce the tumescent solution into surrounding tissue. The larger needle allows the tumescent solution to be injected into the patient more rapidly. Once the desired amount of the tumescent solution is injected into the patient, the medical practitioner removes the needle from the patient. This procedure is repeated for each vein that has been treated with the sclerosing solution. The tumescent solution remains in the surrounding tissue for 12-16 hours after treatment.

The introduction of the tumescent solution around the treated vein in the patient increases compression around the vein and helps maintain the sclerosing agent in the portion of the vein in which it was injected. In addition, the tumescent solution also decreases itching and any pain to the patient. The tumescent solution reduces the inflammation caused by the sclerosing agent and helps dilute any of the sclerosing solution that may inadvertently leak out of the vein. The tumescent solution also decreases the chance of infection and increases homeostasis.

After the tumescent solution has been injected into the patient, the leg is wrapped in compression stockings or bandages for approximately 48 hours and, eventually, blood clotting and scarring occurs that permanently blocks the treated vein. Over time, the vein is reabsorbed by the body, and unsightly lumps flatten out. Blood flow continues through alternative pathways in the patient's venous system.

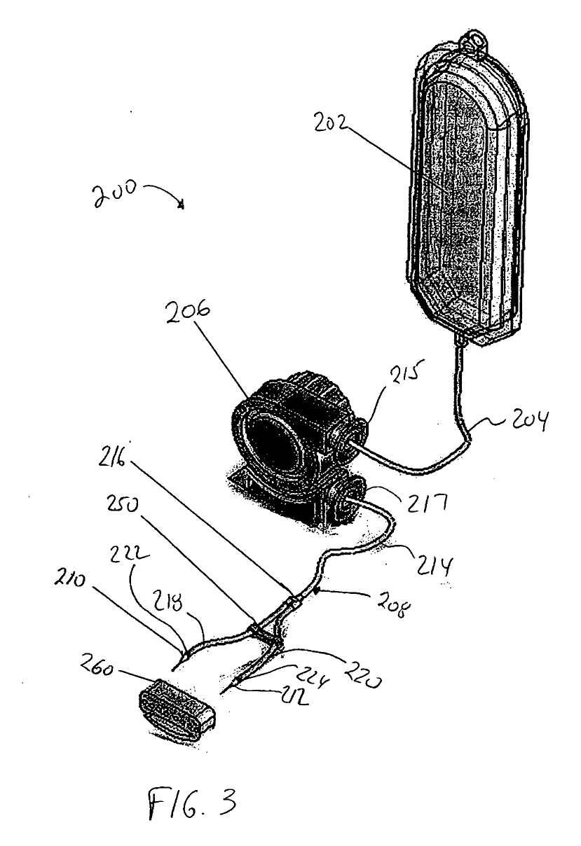

Referring now to FIG. 3, a fluid delivery system 200 for delivering a tumescent solution into a patient is shown. The fluid delivery system 200 preferably includes a fluid source or reservoir 202, a fluid supply line 204, a fluid transmitting device 206, a fluid delivery line 208, and one or more needles. The fluid delivery system 200 delivers fluid or a tumescent solution stored in the fluid source 202 through the fluid transmitting device 206 to the one or more needles. In a preferred embodiment, two needles 210 and 212 are utilized to deliver the tumescent solution into the patient.

The fluid source 202 of the fluid delivery system 200 stores the fluid to be delivered to the patient. The fluid source 202 is preferably a plastic bag, but may be any suitable apparatus for storing or holding fluid. The fluid source 202 can have any suitable size and capacity. The fluid source 202 is coupled to the proximal end of the fluid supply line 204.

The fluid supply line 204 allows fluid to flow from the fluid source 202 to the fluid transmitting device 206. The fluid supply line 204 can be constructed from nylon, Teflon, polyurethane, or polyethylene. It will be recognized that the fluid supply line 204 can be made from a variety of other materials including, for example, polypropylene, polyamide, polyethylenterephthalate, polyamide, other polymers and polycarbonates as well as other suitable forms of plastic. The fluid supply line 204 can have any suitable length depending upon the application and the particular surgical procedure. The distal end of the fluid supply line 204 is coupled to the fluid transmitting device 206.

The fluid transmitting device 206 receives fluid from the fluid source 202 through the fluid supply line 204 and pumps the fluid into the fluid delivery line 208. The fluid transmitting device 206 is preferably a peristaltic pump, but can be any suitable device to pressurize the fluid. The fluid transmitting device 206 preferably has an input port 215 and an output port 217. The input port 215 of the fluid delivery system 206 is coupled to the fluid supply line 204, and the output port 217 is coupled to the fluid delivery line 208. It will be recognized that the fluid transmitting device 206 can have a plurality of input and output ports. The fluid transmitting device 206 can also have controls to permit the medical practitioner to regulate the flow rate of the tumescent solution to the needles 210 and 212 as well as the pressure of the solution.

The fluid delivery line 208 of the fluid delivery system is coupled to the output port 217 of the fluid transmitting device 206. The fluid delivery line 208 can be constructed from any suitable material, such as nylon, plastic or the like. The fluid delivery line 208 is preferably a disposable unit to eliminate resterilization of the line. The fluid delivery line 208 preferably comprises a main supply tube 214, a connector assembly or hub 216, a first delivery tube 218, and a second delivery tube 220. It will be recognized that the fluid delivery line 208 may be a single integrated apparatus or may comprise split tubing.

The proximal end of the main supply tube 214 is connected to the output port 217 of the fluid transmitting device 206 and the distal end of the main supply line 214 is connected to the connector assembly 216. The connector assembly 216 of the fluid delivery line 208 preferably includes an input port, a first output port and a second output port. The input port of the connector assembly 216 is coupled to the distal end of the main supply tube 214. The first output port is coupled to the proximal end of the first delivery tube 218 and the second output port is coupled to the proximal end of the second delivery tube 220. Fluid is transmitted through the main supply tube 214 to the input port of the connector assembly 216 and into the first and second output ports of the connector assembly 216. The fluid then enters the first and second delivery tubes 218 and 220.

The first and second delivery tubes 218 and 220 are relatively soft and flexible so that they can be easily manipulated by a medical practitioner. The first and second delivery tubes 218 and 220 can have any suitable length depending upon the application and the particular surgical procedure. The distal ends of the first and second delivery tubes 218 and 220 are attached to connectors or adapters 222 and 224, respectively. The connectors 222 and 224 can include, but are not limited to, Luer Lock connectors, quick connect fittings, ferrule connectors, threadable connectors, and the like. The connectors 222 and 224 permit the delivery tubes 218 and 220 to be attached or coupled to the needles 210 and 212. In addition, clamps or stop cocks (not shown) may also be attached to the delivery tubes 218 and 220 to regulate or interrupt the flow of fluid through the lumens of the delivery tubes.

As shown in FIG. 3, a separator 250 can be coupled to the first and second delivery tubes to hold the first delivery line at a certain distance from the second delivery line. A holding apparatus 260 can also be utilized to hold the needles of the fluid delivery system at a desired location during the procedure. The holding apparatus 260 preferably has a plurality of holes to insert the needles of fluid delivery device 200. A weight (not shown) can also be attached to the first and second delivery lines to hold the lines at a desired position and prevent the lines from moving during the procedure.

The apparatus and methods of the present invention allow a medical practitioner to treat undesired veins in a body of a patient. The surgical apparatus allows the medical practitioner to introduce fluid, such as saline, into the tissue where the vein has been treated by a sclerosing agent. The introduction of the tumescent solution into the tissue causes the surrounding tissue to become swollen and firm, thereby compressing the vein and maintaining the solution in the portion of the vein in which it was injected. As a result, the effect of the sclerosing solution is increased. The treated veins disappear in less time and the patient has a faster recovery time with improved results. The tumescent solution also reduces hyper-pigmentation and inflammation of the tissue surrounding the vein and decreases any itching and pain to the patient. This procedure further reduces the amount of time that patient has to wear support hosiery and decreases the number of treatment sessions for the patient, thereby reducing the cost to the patient. The present invention also allows the medical practitioner to efficiently deliver the tumescent solution around the vein during the sclerotherapy procedure.

Although the present invention has been described in detail by way of illustration and example, it should be understood that a wide range of changes and modifications can be made to the preferred embodiments described above without departing in any way from the scope and spirit of the invention. For example, a fiber optic visualization apparatus can be incorporated into any of the surgical apparatus described above.

Thus, the described embodiments are to be considered in all respects only as illustrative and not restrictive, and the scope of the invention is, therefore, indicated by the appended claims rather than the foregoing description. All changes that come within the meaning and range of equivalency of the claims are to be embraced within their scope.

I claim:

1. A method of treating an undesired vein in a human body, the method comprising:
inserting a needle of a syringe into a segment of the undesired vein;
injecting a sclerosing agent through the needle into the segment of the undesired vein to initiate occlusion of the vein;
removing the needle of the syringe from the undesired vein;
inserting a needle of a fluid delivery system into tissue surrounding the undesired vein;
delivering a tumescent solution through the needle of the fluid delivery system into the surrounding tissue of the vein to increase compression to the undesired vein.

2. The method of claim 1 where the undesired vein includes one of a varicose vein and a spider vein.

3. The method of claim 1 further comprising the steps of removing the needle of the fluid delivery system, inserting another needle of a fluid delivery device into the surrounding tissue of the undesired vein and delivering a tumescent solution through the needle into the surrounding tissue of the vein to increase compression to the undesired vein.

4. The method of claim 4 wherein the needles of the fluid delivery system have different sizes.

5. A fluid delivery system to deliver fluid in a body of a patient, the system comprising:
a fluid source to store the fluid;
a fluid transmitting device in fluid communication with the fluid source;
a fluid delivery tube, having a first and a second lumen, coupled to the fluid transmitting device;
a first needle in fluid communication with the first lumen; and
a second needle in fluid communication with the second lumen.

6. The fluid delivery system of claim 5 wherein the fluid source comprises a plastic bag.

7. The fluid delivery system of claim 5 wherein the fluid transmitting device comprises a pump.

8. The fluid delivery system of claim 5 wherein the fluid delivery tube comprises split tubing having a first delivery tube and a second delivery tube.

9. The fluid delivery system of claim 5 wherein the fluid delivery tube comprises:
a main supply tube coupled to a connector assembly;
a first delivery tube coupled to the connector assembly; and
a second delivery tube coupled to the connector assembly.

10. The fluid delivery system of claim 5 wherein the first needle has a smaller diameter than the second needle.

11. The fluid delivery system of claim 8 further comprising a separator coupled to the first and the second delivery tubes.

12. The fluid delivery system of claim 9 further comprising a separator coupled to the first and the second delivery tubes.

13. The fluid delivery system of claim 9 further comprising a holder to support the first and the second needles.
